(19) United States
(12) Patent Application Publication
(10) Pub. No.: US 2002/0085665 A1
(43) Pub. Date: Jul. 4, 2002

(54) High density flex interconnect for CT detectors

(76) Inventors: David M. Hoffman, New Berlin, WI (US); Bing Shen, Cary, NC (US)

Correspondence Address: John S. Beulick, Armstrong Teasdale LLP, One Metropolitan Sq., Suite 2600, St. Louis, MO, 63102, US

(21) Appl. No.: 09/751,824

(22) Filed: Dec. 29, 2000

(51) Int. Cl. A61B 6/00

(52) U.S. Cl. 378/19; 378/4; 250/363.01

(57) ABSTRACT

An enhanced photosensor module is used in a computed tomography system having a DAS system for receiving data. The module includes a substrate having a photodiode array thereon optically coupled to a scintillator array, an FET chip electrically connected to the photodetector system via a flex connector and mounted on the substrate. The module also includes a flex circuit connected to the FET chip and the DAS system. The flex circuit is mounted on the substrate and positioned 90 degrees to the substrate.

### BACKGROUND OF THE INVENTION

This invention relates generally to radiation detectors of the scintillating type, and more particularly to a high density flex interconnect system for computer tomograph CT detectors and to methods for preparing and using the interconnect system.

In at least one known computed tomography (CT) imaging system configuration, an x-ray source projects a fan-shaped beam which is collimated to lie within an X-Y plane of a Cartesian coordinate system and generally referred to as the “imaging plane”. The x-ray beam passes through the object being imaged, such as a patient. The beam, after being attenuated by the object impinges upon an array of radiation detectors. The intensity of the attenuated beam radiation received at the detector array is dependent upon the attenuation of the x-ray beam by the object. Each detector element of the array produces a separate electrical signal that is a measurement of the beam attenuation at the detector location. The attenuation measurements from all the detectors are acquired separately to produce a transmission profile.

In known third generation CT systems the x-ray source and the detector array are rotated with a gantry within the imaging plane and around the object to be imaged so that the angle at which the x-ray beam intersects the object constantly changes. A group of x-ray attenuation measurements, i.e., projection data, from the detector array at one gantry angle is referred to as a “view”. A “scan” of the object comprises a set of views made at different gantry angles, or view angles, during one revolution of the x-ray source and detector. In an axial scan, the projection data is processed to construct an image that corresponds to a two dimensional slice taken through the object. One method of reconstructing an image from a set of projection data is referred to in the art as the filtered back projection technique. This process converts the attenuation measurements from a scan into integers called “CT numbers” or “Hounsfield” units which are used to control the brightness of a corresponding pixel in a cathode ray tube display.

At least one known detector in CT imaging systems comprises a plurality of detector modules, each having a scintillator array optically coupled to a semiconductor photodiode array that detects light output by the scintillator array. semiconductor photodiode array that detects light output by the scintillator array. These known detector module assemblies require an adhesive bonding operation to assemble. The photodiode array and scintillator must be accurately aligned with an alignment system, using a plastic shim to set a gap between the photodiode and scintillator arrays. After alignment, the four corners of the assembly are tacked together with an adhesive to hold the alignment. The tack is cured, and the thin gap between the photodiode and scintillator arrays is filled by dipping the assembly into an optical epoxy adhesive, which wicks into the entire gap. The epoxy is cured, and the scintillator is thus epoxied to the diode array.

### BRIEF SUMMARY OF THE INVENTION

There is therefore provided, in one embodiment of the present invention, an improved high density interconnect system (for a CT detector module) comprising a flat sheet having a rectangular cross section. Advantageously, in an embodiment, this invention utilizes a flex circuit whose longitudinal axis is at 90 degrees to the axis of the diode array.

Among other advantages, this improved high density interconnect system provides for a significant relaxation of (increase in) the interconnect flex density from both the CT detector diode assembly to the flex circuit and the flex run density thereby enabling expansion of the current design to larger coverage meaning more slices, and high resolution meaning smaller cell sizes which enables new higher density/larger detector arrays.

In addition, this and other embodiments of the invention provide various combinations of additional advantages, including reducing the flex run pitch densities which allows cost effective expansion of the interconnect density within the current state of the art limits.

### BRIEF DESCRIPTION OF THE DRAWINGS

FIG. 1 is a pictorial view of a CT imaging system.

FIG. 2 is a block schematic of the system illustrated in FIG. 1.

FIG. 3 is a perspective view of one embodiment of a CT system detector array of the present invention.

FIG. 4 is an overview of the flex high density interconnect.

FIG. 5 is an overview of the flex high density interconnect depicting an extended diode concept with diodes butted.

FIG. 6 is an overview of the flex high density interconnect depicting an extended FET concept.

FIG. 7 is an overview of the flex high density interconnect having an FET built onto diode concept, silicon concept and multilayer printed wiring assembly concept.

FIG. 8 is an overview of the flex high density interconnect having a separate FET concept, a multilayer PWA concept and a metal on silicon chip.

FIG. 9 is an overview of the flex high density interconnect depicting a 32 slice diode array.

FIG. 10 is an overview of the flex high density interconnect depicting an eight slice diode array.

FIG. 11 is an overview of the flex high density interconnect depicting a multilayer ceramic diode array.

### DETAILED DESCRIPTION OF THE INVENTION

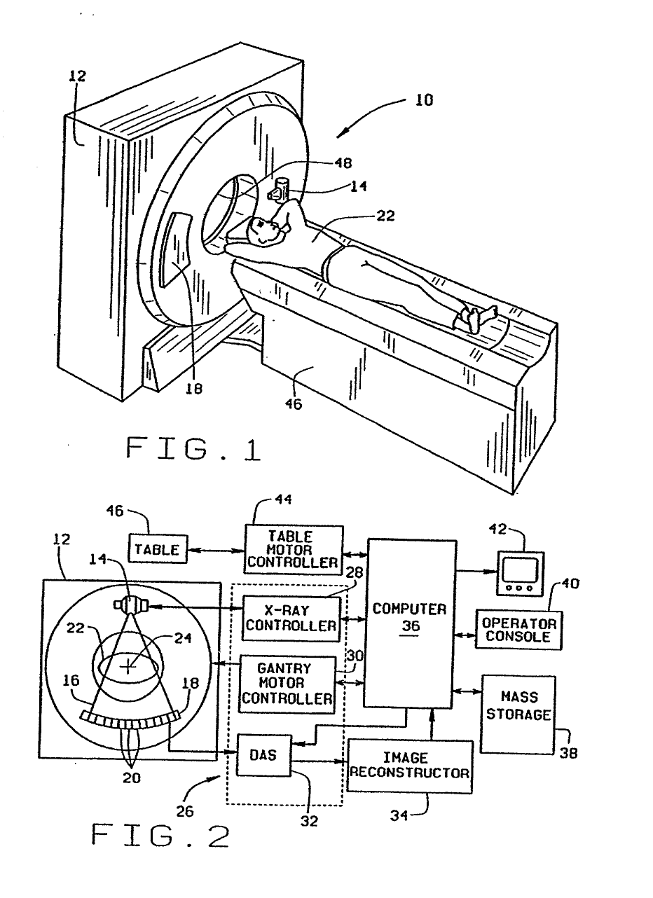

Referring to FIGS. 1 and 2, a computed tomography (CT) imaging system 10 is shown as including a gantry 12 representative of a third generation CT scanner. Gantry 12 has an x-ray source that 14 that projects a beam of x-rays 16 toward a detector array 18 on opposite side of gantry 12. Detector array 18 is formed by detector elements 20 which together sense the projected x-rays that passes through an object 22, for example a medical patient. Each detector element 20 produces an electrical signal that represents the intensity of an impinging x-ray beam and hence the attenuation of the beam as it passes through patient 22. During a scan to acquire x-ray projection data, gantry 12 and the components mounted thereon rotate about a center of rotation 24. Detector array 18 may be fabricated in a single slice or multi-slice configuration. In a multi-slice configuration, detector array 18 has a plurality of rows of detector elements 20, only one of which is shown in FIG. 2.

Rotation of gantry 12 and the operation of x-ray source 14 are governed by a control mechanism 26 of CT system 10. Control mechanism 26 includes an x-ray controller 28 that provides power and timing signals to x-ray source 14 and a gantry motor controller 30 that controls the rotational speed and position of gantry 12. A data acquisition system (DAS) 32 in control mechanism 26 samples analog data from detector elements 20 and converts the data to digital signals for subsequent processing. An image reconstructor 34 receives sampled and digitized x-ray data from DAS 32 and performs high speed image reconstruction. The reconstructed image is applied as an input to a computer 36 which stores the image in a mass storage device 38.

Computer 36 also receives commands and scanning parameters from an operator via console 40 that has a keyboard. An associated cathode ray tube display 42 allows the operator to observe the reconstructed image and other data form compute 36. The operator supplied commands and parameters are used by computer 36 to provide control signals and information to DAS 32, x-ray controller 28 and gantry motor controller 30. In addition, computer 36 operates a table motor controller 44 which controls a motorized table 46 to position patient 22 in gantry 12. Particularly, table 46 moves portions of patient 22 through gantry opening 48.

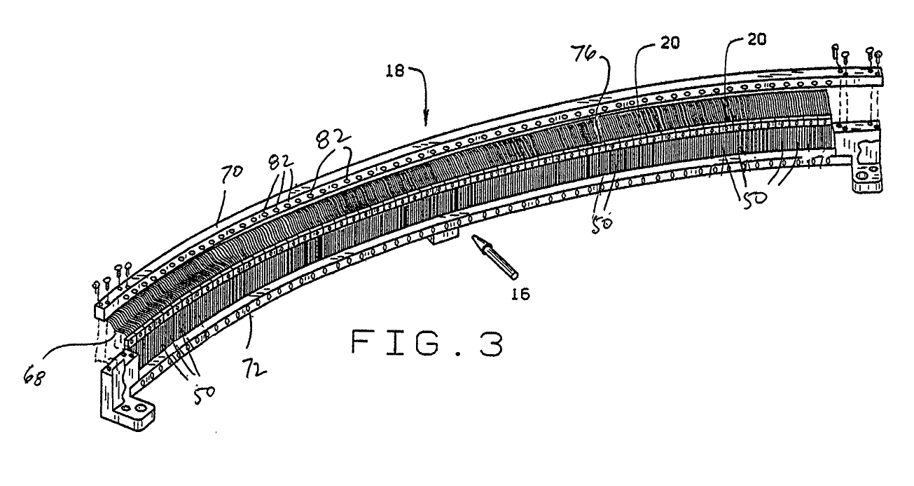

As shown in FIG. 3, detector array 18 includes a plurality of detector module assemblies 50 (also referred to as detector modules), each module comprising an array of detector elements 20. Each detector module 50 includes a high-density photosensor array (not shown) and a multidimensional scintillator array (not shown) positioned above and adjacent to photosensory array (not shown). Particularly, scintillator array (not shown) includes a plurality (not shown), while photosensor array (not shown) includes photodiodes (not shown), a switch apparatus (not shown) and a decoder (not shown). Photodiodes 58 are individual photodiodes. In another embodiment, photodiodes 58 are deposited or formed on a substrate. Scintillator array 54, as known in the art, is positioned over or adjacent photodiodes 58. Photodiodes 58 are optically coupled to scintillator array 54 and have electrical output lines for transmitting signals representative of the light output by scintillator array 54. Each photodiode 58 produces a separate low level analog output signal that is a measurement of beam attenuation for a specific scintillator of scintillator array 54. Photodiode output lines (not shown in FIG. 3) may, for example, be physically located on one side of module 20 or on a plurality of sides of module 20. In another embodiment (not shown), photodiode outputs are located at opposing sides of the photodiode array.

In one embodiment, as shown in FIG. 3, detector array 18 includes fifty-seven detector modules 50. Each detector module 50 includes a photosensor array 52 and scintillator array 54, each having a detector element 20 array size of 16×16. As a result, array 18 is segmented into 16 rows and 912 columns (16×57 modules ) allowing up to N=16 simultaneous slices of data to be collected along a z-axis with each rotation of gantry 12, where the z-axis is an axis of rotation of the gantry.

Switch apparatus (not shown) is a multidimensional semiconductor switch array. Switch apparatus (not shown) is coupled between photosensor array (not shown) and DAS 32. Switch apparatus (not shown), in one embodiment, includes two semiconductor switch arrays (not shown). Switch arrays (not shown) each include a plurality of field effect transistors (FETS) (not shown) arranged as a multidimensional array. Each FET includes an input electrically connected to one of the respective photodiode output lines, an output, and a control (not shown) arranged as a multidimensional array. Each FET includes an input electrically connected to one of the respective photodiode output lines, an output, and a control (now shown). FET outputs and controls are connected to lines that are electrically connected to DAS 32 via a flexible electrical cable (not shown). Particularly, about one-half of the photodiode output lines are electrically connected to each FET input line of switch (not shown) with the other one-half of photodiode output lines electrically connected to DAS 32 via a flexible electrical cable (not shown). Particularly about one-half of the photodiode output lines are electrically connected to each FET input line of switch (not shown) with the other one-half of photodiode output lines electrically connected to FET input lines of switch (not shown). Flexible electrical cable (not shown) is thus electrically coupled to photosensor array 52 and is attached, for example, by wire bonding.

Decoder 62 controls the operation of switch apparatus 60 to enable, disable, or combine photodiode 58 outputs depending upon a desired number of slices and slice resolution for each slide. Decoder (not shown) in one embodiment, is an FET controller as known in the art. Decoder (not shown) includes a plurality of output and control lines coupled to switch apparatus (not shown) and DAS 32. Particularly, the decoder outputs are electrically coupled to the switch apparatus control lines to enable switch apparatus (not shown) to transmit the proper data from the switch apparatus inputs to the switch apparatus outputs. Utilizing decoder (not shown), specific FETS within switch apparatus (not shown) are selectively enabled, disable, or combine so that specific photodiode 58 outputs are electrically connected to CT system DAS 32. Decoder (not shown) enables switch apparatus (not shown) so that a selected number of rows of photosensor array 52 are connected to DAS 32, resulting in a selected number of slices of data being electrically connected to DAS 32 for processing.

As shown in FIG. 3, detector modules 50 are filled in a detector array 18 and secured in place by rails 70 and 72, FIG. 3 shows rail 72 already secured in place, while rail 70 is about to be secured over electrical cable (not shown), over module 50 substrate (not shown), flexible cable (not shown), and mounting bracket 76. Screws (not shown in FIG. 3) are then threaded through holes (not shown) and into threaded holes (not shown) of rail 70 to secure modules 50 in place. Flanges (not shown) of mounting brackets 76 are held in place by compression against rails 70 and 72 (or by bonding, in one embodiment) and prevent detector modules 50 from “rocking”. Mounting brackets 76 also clamp flexible cable (not shown) against substrate 74, in one embodiment, flexible cable (not shown) is also adhesively bonded to substrate (not shown).

A flex circuit is used which is rotated 90 degrees to the current design. This rotation allows the use of wider flexes thereby reducing the flex run pitch densities. The flex herein is a rectangular flat sheet having a rectangular cross section. The flex circuit is thin to allow it to be sandwiched between adjacent detector modules. In an alternative embodiment, the ceramic has a step in the area of the flex to eliminate the need for a thinned flex. In an exemplary embodiment, the ceramic has radial edges to reduce the flex bend radius. In one embodiment, the diode array is a single piece or two pieces butted in the center of the array. The diode is extended under the flex and contains a 2 D array of interconnects. This design includes the FET array as a chip on the flex. In another embodiment, the FET array is included in the DAS or the design could have a single DAS channel per detector cell. The diode could also still be wire bonded to an FET chip and then this chip could be extended under the flex and incorporate a 2 D array of interconnects. The diode could also be wire bonded to a separate Silicon chip that extends under the flex circuit accomplishing the same effect. A ceramic with a step could be utilized in both of the latter approaches. The connection from this 2D set of interconnects to the flex is accomplished in a number of ways. These include a ball grid array with a solder reflow process, a set of pads above the flex, a two dimensional fine pitch elastomer interposer or thermal bonding with an ACF film.

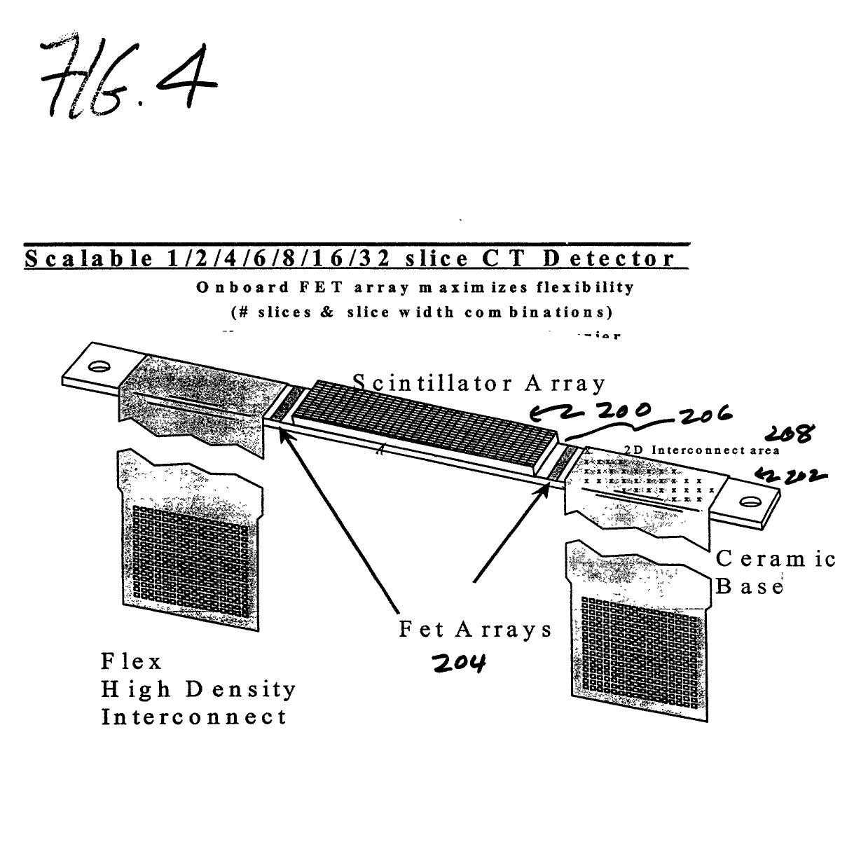

FIGS. 4-11 depict embodiments of this high density flex interconnect for CT detectors. The modules (depicted in FIGS. 4-11) are connected to the detector array 18 of FIG. 3 by mechanically affixing the module to the array through bolting or otherwise mechanically affixing the module to the array. Holes are provided on the substrate of the module for bolting. The flex high density interconnect is electrically connected to the DAS by attaching the flex to the DAS system whereby electrical signals from the detector module are transmitted to the DAS system via the interconnect and the circuit.

In a perspective view in FIG. 4, a high density flex interconnect for CT detectors is depicted having a scintillator array 200 bonded on a ceramic base substrate 202 with FET 204 arrays likewise mounted on the substrate 202 but at a gap distance 206 from the scintillator 208. A large area two dimensional interconnect 208 is depicted at the one respective end of this module, the interconnect 208 having a large two dimensional contact area and is rotated at a 90 degree angle to the ceramic base. The interconnect 208 is attached to the ceramic base 202.

The interconnect is affixed to the ceramic base is by one of a solder reflux, an anisotropic conductive film (ACF) or an elastomeric connector with a clamp. In an alternative embodiment, a bumps and dimples contact is used.

The modules depicted in FIGS. 4-11 are connected to the detector array 18 of FIG. 3 by mechanically affixing the module to the array. Holes are provided on the substrate of the module. The flex high density interconnect is electrically connected to the DAS by attaching the flex to the DAS system whereby electrical signals from the detector module are transmitted to the DAS system.

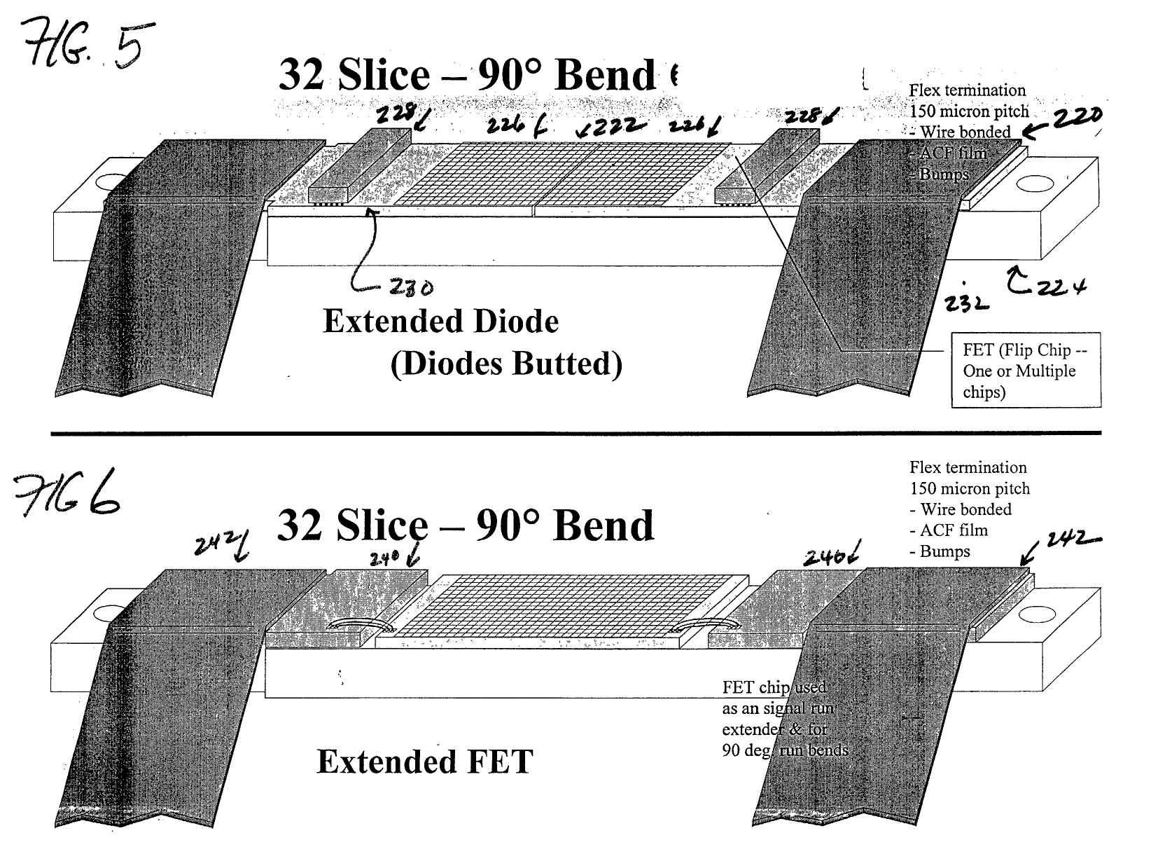

FIG. 5 depicts a CT module having a high density flex interconnect 220, where the module has a photodiode chip 220 mounted on a substrate 224 with a diode array 226 cut in half. (This diode does not have to be cut, but it is depicted this way merely to show the allowable use of wafer chips (6 inch) which are available today.) Two FET chips 228 are mounted directly on the silicon chip 230 (“flip chip design”) one at each end of the module. A silicon chip extends under the flex atop the ceramic base 224. The flex circuit 232 bends 90 degrees off of each side. The two dimensional array interconnect 220 is located on the underside of the flex and on the top of the silicon. This connection uses solder reflow, or an anisotropic conductive film (ACF) or an elastomer connection with clamps or a bumps and dimples connection.

FIG. 6 depicts a module similar to that depicted in FIG. 6 with the diode chip unchanged; however, in FIG. 6 the FET chip 240 extends under the flex 242. The FET chip 240 is used as a signal run extender and for 90 degree run bends. A cutout is shown on the left side of the module for purposes of illustrating the 90 degree bend.

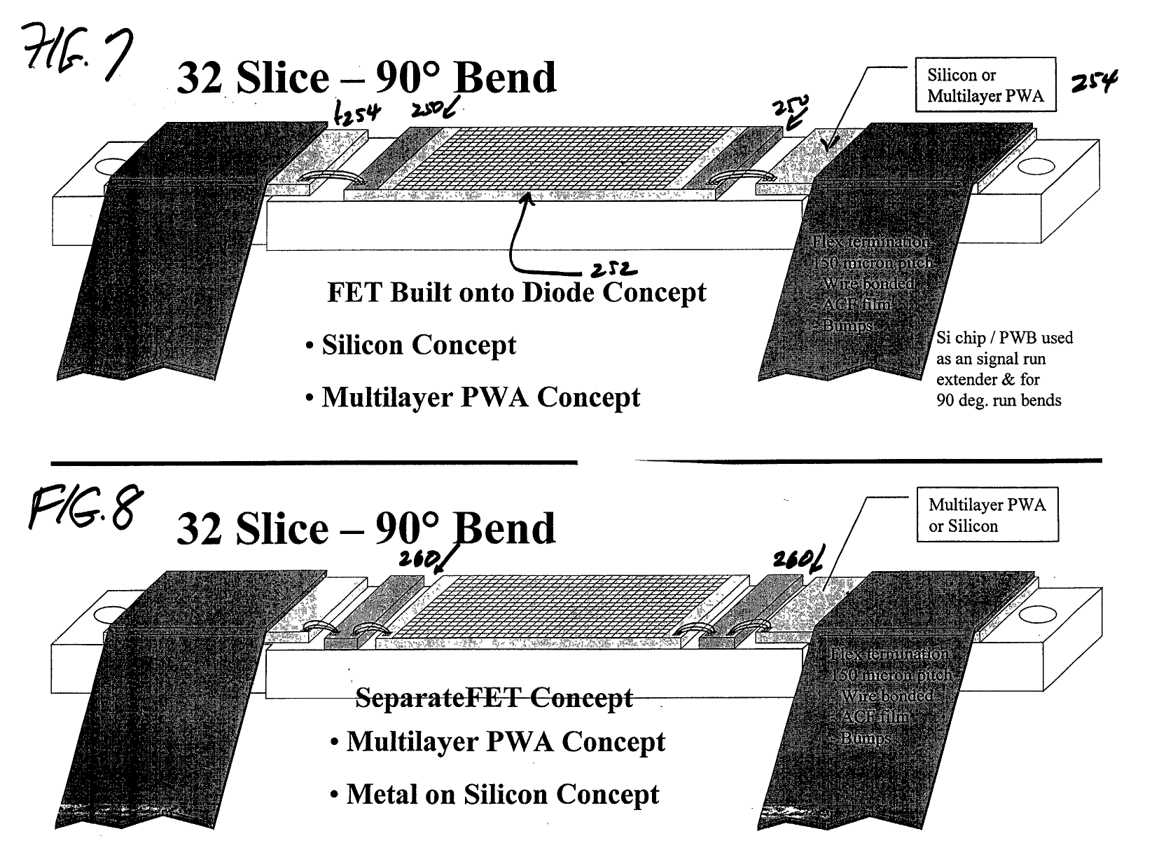

FIG. 7 depicts a module similar to that depicted in FIG. 7; however, this embodiment depicts the FET chip 250 being built as part of the diode chip 252, A separate silicon connector chip 254 with no active circuitry extends under the high density interconnect. A printed wiring assembly (not shown) may be employed as an alternative to a silicon chip. The silicon chip or PWB is used as a signal run extender and for 90 degree run bends.

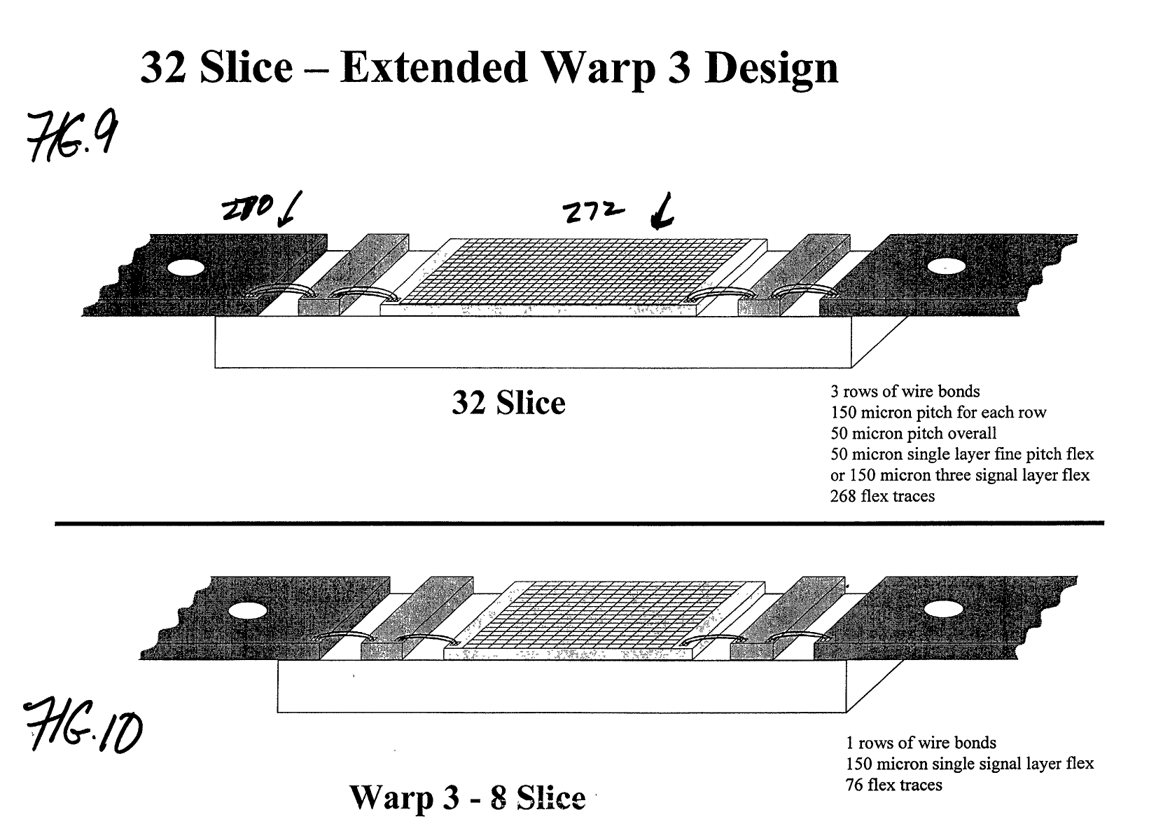

FIG. 8 depicts a module similar to that depicted in FIG. 8 but separate FET switches 260 are provided. These separate FET switches could be glued onto the flex itself.

FIG. 9 depicts a module similar to that depicted in FIG. 9; however, the flex 276 has multiple metal layers and is copper shielded. The width is the width of the module as modules in this embodiment are butted together. The substrate is ceramic. Diode 272 is a two dimensional array.

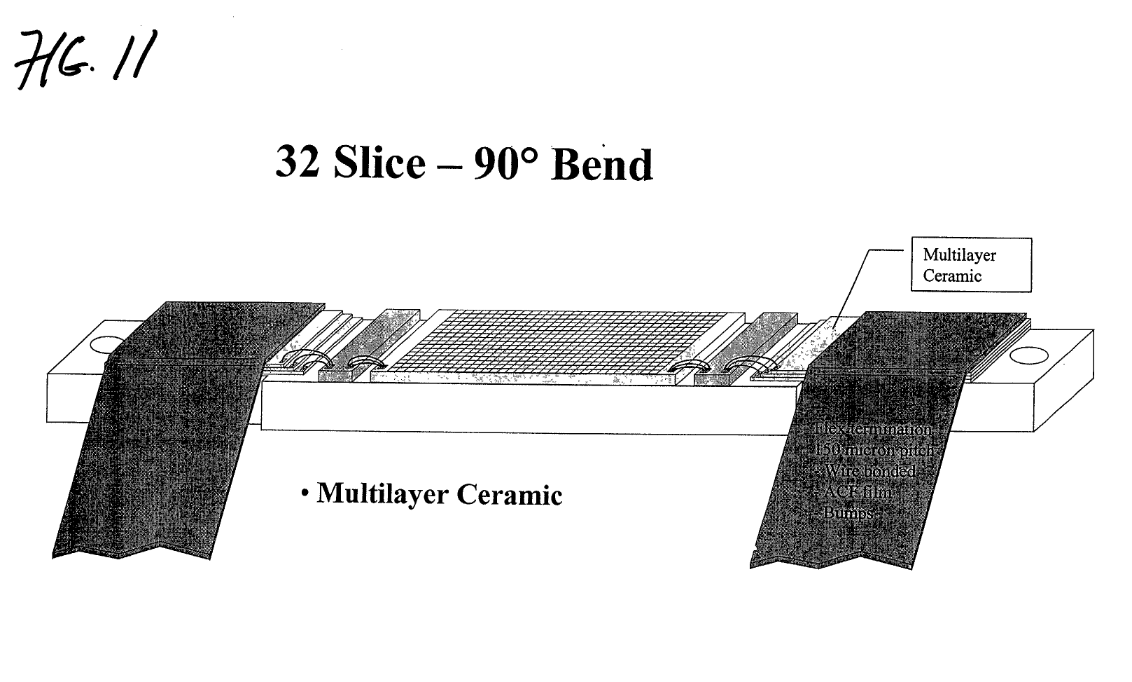

FIG. 10 depicts a module similar to that depicted in FIG. 10; however, it is substantially the same as that depicted in FIG. 9 with the embodiments shown as having one row of wire bonds, a 150 micro single signal layer flex and 76 flex traces.

FIG. 11 depicts a module similar to that depicted in FIG. 9; however, a multilayer ceramic is depicted.

In making the above embodiments, bonding of the stintillator, diodes, FET chips, and high density flex interconnect to a silicon chip and/or ceramic base is carried out by methods known to those of skill in the art.

While the invention has been described in terms of various specific embodiments, those skilled in the art will recognize that the invention can be practiced with modification within the spirit and scope of the claims.

What is claimed is:

1. An enhanced photosensor module for use in a computed tomography system having a DAS system for receiving data, said module comprising a substrate having a photodiode array theron optically coupled to a scintillator array, an FET chip electrically connected to said photodetector system via a flex connector and mount on said substrate, a high density interconnect, and a flex circuit connected to said FET chip and said DAS system, said flex interconnect is mounted on said substrate and is a flat sheet having a rectangular cross sectional area.

2. A photosensor in accordance with claim 1 wherein substrate comprises a cut diode chip to accommodate a flex circuit.

3. A photosensor in accordance with claim 2 wherein the sides of said chip are stepped to accommodate a flex circuit.

4. A photosensor in accordance with that of claim 3 wherein the longitudinal axis of the flex circuit is perpendicular to the horizontal axis of the substrate and diode.

5. A photosensor in accordance with that of claim 4 wherein the longitudinal axis of the flex circuit is bent 90 degrees to the horizontal axis of the substrate.

6. An improved high density interconnect system of a CT detector module associate with a DAS system, wherein said detector system comprises a photodiode array mounted on the substrate and optically connected with a scintillator array, an FET chip mounted on said substrate and electrically connected to said photodiode array and a flex circuit electrically connecting said FET chip to a DAS system wherein the flex connector is a flat sheet having a rectangular cross sectional area.

7. A high density interconnect system in accordance with claim 6 wherein the substrate comprises a cut diode chip.

8. A high density interconnect system in accordance with claim 6 wherein the sides of said chip are stepped to accommodate a flex circuit.

9. A high density interconnect system in accordance with that of claim 6 wherein the longitudinal axis of the flex circuit is perpendicular to the horizontal axis of the substrate and diode.

10. A high density interconnect system in accordance with that of claim 6 wherein the longitudinal axis of the flex circuit is bent 90 degrees to the horizontal axis of the substrate.

11. A method of making an improved high density interconnect system for a CT detector module having a flex connection which is a flat sheet having a rectangular cross sectional area which comprises:
affixing a photodiode array on a substrate and optically coupling a scintillator array therewith;
affixing an FET chip on said substrate and electrically connecting said FET chip to said photodiode array and
attaching as the flex circuit electrical connector to said FET chip and to a DAS system, a flat sheet having a rectangular cross sectional area.

12. A method in accordance with that of claim 11 wherein a high density interconnect is used to connect said FET chip with said flex circuit and said interconnect is a flat sheet having a rectangular cross sectional area.

13. A method in accordance with that of claim 11 wherein said interconnect is a flat sheet of metal.

14. A method in accordance with that of claim 11 wherein said flat sheet is affixed to said chip via bonding.

15. An enhanced computed tomograph (CT) system which comprises an enhanced photosensor module for use in the computed tomography system having a DAS system for receiving data, said module comprising a substrate having a photodiode array thereon optically coupled to a scintillator array, an FET chip electrically connected to said photodetector system via a flex connector and mounted on said substrate, and a flex circuit electrically connected to said FET chip and said DAS system, wherein said flex circuit is a flat sheet having a rectangular cross sectional area.

16. An enhanced compouted tomograph (CT) in accordance with that of claim 16 wherein said flex connector has a rectangular cross sectional area.

17. An enhanced computed tomograph (CT) in accordance with that of claim 17 wherein said flex circuit has its longitudinal axis at 90 degrees to the horizontal axis of said substrate.
